(19) United States
(12) Patent Application Publication
(10) Pub. No.: US 2012/0245619 A1
(43) Pub. Date: Sep. 27, 2012

(54) Retrievable Filter with Retractable Wire Barbs and Method of Use

(75) Inventors: Erin Guest, Minneapolis, MN (US); Chad Perrin, Coon Rapids, MN (US); Burns Doran, Monticello, MN (US)

(73) Assignee: BOSTON SCIENTIFIC SCIMED, INC., Maple Grove, MN (US)

(21) Appl. No.: 13/424,466

(22) Filed: Mar. 20, 2012

Related U.S. Application Data
(60) Provisional application No. 61/467,675, filed on Mar. 25, 2011.

(51) Int. Cl. A61F 2/01

(52) U.S. Cl. 606/200

(57) ABSTRACT

The disclosure relates to retrievable filter devices implantable within a lumen. The retrievable filters include retractable anchoring barbs. Associated apparatus and methods for delivering, retrieving, and/or repositioning the filter are also disclosed herein.

### BACKGROUND

Blood clot filters are used in combination with other thrombolytic agents to treat pulmonary embolism occurring within a patient. Such devices are generally inserted intravenously into a target location of the body (e.g. an artery or vein), and function by capturing blood clots (emboli) contained in the blood stream before they can reach the heart and/or lungs and cause permanent damage to the body. In the treatment of Deep Vein Thrombosis (DVT), for example, such filters are placed in the inferior vena cava to prevent further blood clotting in the large veins of the lower body. Placement of the filter is typically accomplished percutaneously via the femoral arteries or the jugular vein using a local anesthetic, or by performing a laparotomy with the patient under general anesthesia.

In certain designs, an introducer sheath may be used to deliver the blood clot filter through the body. Such introducer sheaths are generally tubular in shape, and include an inner lumen configured to transport the blood clot filter in a collapsed position through the body. Once transported to a desired location within the vasculature, the filter can then be removed from within the introducer sheath, allowing the filter to spring open, or to be expanded, and engage the vessel wall. A needle, hook, barb, prong, wedge or other attachment means disposed on the blood clot filter can be used to secure the filter to the vessel wall.

There are a number of situations in which it may be desirable for a physician to remove the filter once inserted within the body. In certain circumstances, for example, the risk of pulmonary embolism may be relatively short term (e.g. about two weeks), thus requiring insertion of the filter for only a short period of time. Permanent implantation of the filter in such cases may unnecessarily impede the flow of blood within the vessel, and can lead to further thrombosis growth at the filter implantation site. In other circumstances, it may be desirable to reposition the filter within the vessel, or to replace the existing filter with a new filter.

### SUMMARY

This disclosure pertains generally to retrievable filter devices implantable within a lumen. Associated deployment/retrieval apparatus and methods for retrieving and/or repositioning the filter device within the body are also disclosed herein.

In some embodiments, this disclosure relates to an implantable filter having a first generally apical hub, a first plurality of solid legs each having a proximal end, a distal end region, and an intermediate region therebetween, said first plurality of solid legs being fixedly attached to the first generally apical hub and having a first configuration in which the solid legs are substantially parallel and a second configuration in which the distal ends of at least some the first plurality of solid legs are radially expanded relative to a central axis passing through the first generally apical hub; and a second plurality of wires each having distal ends and proximal ends, said wires of the second plurality of wires being fixedly attached at their proximal ends to a second generally apical hub which is slidably moveable relative to the first generally apical hub, wherein each of the second plurality of wires is individually associated with one of the solid legs of the first plurality of solid legs at the intermediate region thereof and distal end region thereof, further wherein the association of each wire with the solid leg at the distal end region thereof comprises the wire passing transversely through the distal end region of the solid leg such that each wire has a first position in which the distal end of the wire protrudes radially outward from the distal end region of the solid leg and a second position in which the wire does not protrude significantly radially outward from the distal end region of the solid leg.

In other embodiments, this disclosure relates to an implantable filter comprising a generally apical hub; a plurality of legs each having a proximal end, a distal end region, and an intermediate region therebetween, said legs being fixedly attached to the generally apical hub and having a first configuration in which the legs are substantially parallel and a second configuration in which the distal ends of at least some the plurality of legs are radially expanded relative to a central axis passing through the apical hub; a pair of first struts attached to a first leg and a second leg of the plurality of legs within the respective intermediate regions of the first and second legs and extending to a first join proximal to their distal ends; a pair of second struts attached to a first leg and a second leg within the respective intermediate regions of the first and second legs and extending to a second join distal to their proximal ends; and a wire extending from the first join and slidingly received through an aperture associated with the second join, wherein upon radial expansion of the first and second leg to the second configuration the first join and the second join are configured to move generally toward one another whereby the wire protrudes from the second join in a direction radially outward from the central axis passing through the apical hub.

In another aspect, this disclosure relates to method of deploying a filter comprising positioning the filter within a distal region of a catheter; positioning the distal region of the catheter within a lumen containing a fluid to be filtered; ejecting the filter from the catheter thereby allowing at least some the distal ends of the plurality of solid legs to become radially expanded relative to a central axis passing through the apical hub; and implanting the distal ends of the plurality of wires in a wall of the lumen containing a fluid to be filtered.

In yet another aspect, this disclosure relates to a method of retrieving a filter comprising positioning a distal region of a catheter proximally adjacent to a first generally apical hub of the filter; moving the first generally apical hub of the filter axially relative to a second generally apical hub, thereby withdrawing a plurality of wires relative to a plurality of solid legs thereby moving the plurality of wires from first position in which the wires protrude significantly radially outward from the distal end region of the solid legs to a second position in which the distal ends of the wires do not significantly protrude radially outward from the distal end region of the solid legs thereby at least partially withdrawing the wire from the wall of the lumen; withdrawing the filter to a position at least partially within a distal region of the catheter; and withdrawing the catheter and the filter of claim 1 from the lumen.

Although described herein in the context of a temporary vena cava filter, it will be understood that the anchorage system disclosed may be used to provide permanent or temporary anchorage for a variety of endoluminal devices which are not necessarily limited to blood vessels.

### BRIEF DESCRIPTION OF DRAWINGS

FIG. 1 illustrates an embodiment of the implantable filter.

FIG. 1A illustrates a detail of the implantable filter rotated 90 degrees.

FIG. 2 illustrates the implantable filter of FIG. 1 in a second position.

FIGS. 3-6, 7, 7A, 8 and 8A illustrate a number of alternate configurations of the distal ends of the legs of the implantable filter of FIGS. 1 and 2.

FIGS. 9-12 illustrate alternate configurations of the intermediate region of the legs of the implantable filter of FIGS. 1 and 2.

FIG. 13 illustrates an alternate configuration of a leg of the implantable filter of FIGS. 1 and 2.

FIG. 14 illustrates an embodiment of the implantable filter.

FIG. 15A illustrates a fragmentary representation of an embodiment of the implantable filter in a first configuration.

FIG. 15B illustrates the implantable filter of FIG. 15A in a second configuration.

FIG. 16 is a detail of an implantable filter delivery and retrieval system.

### DETAILED DESCRIPTION

The following description should be read with reference to the drawings wherein like reference numerals indicate like elements throughout the several views. The drawings, which are not necessarily to scale, are not intended to limit the scope of the claimed invention. The detailed description and drawings illustrate example embodiments of the claimed invention.

All numbers are herein assumed to be modified by the term “about.” The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, “an”, and “the” include the plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect such feature, structure, or characteristic in connection with other embodiments, whether or not explicitly described, unless cleared stated to the contrary.

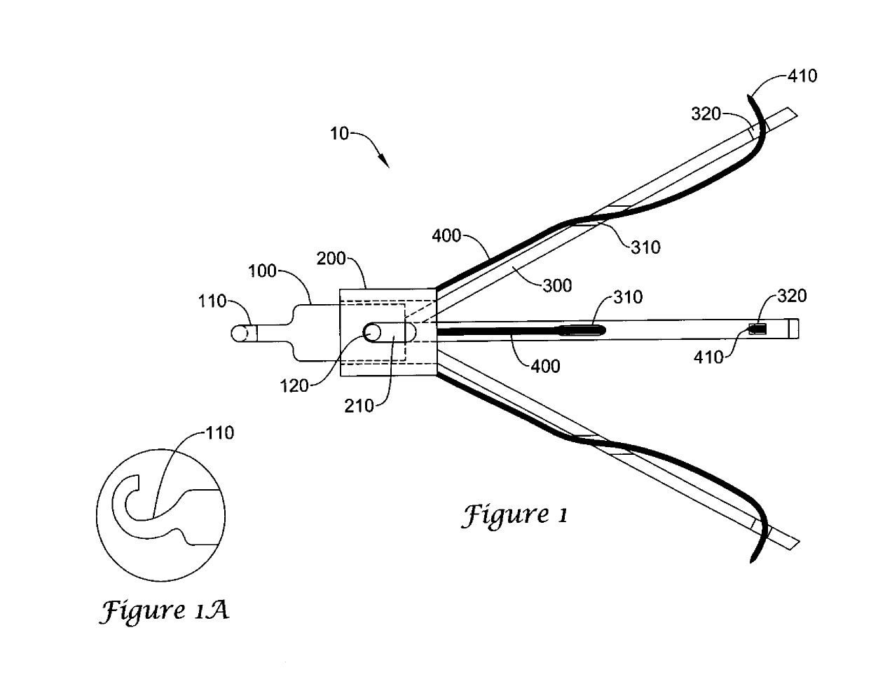

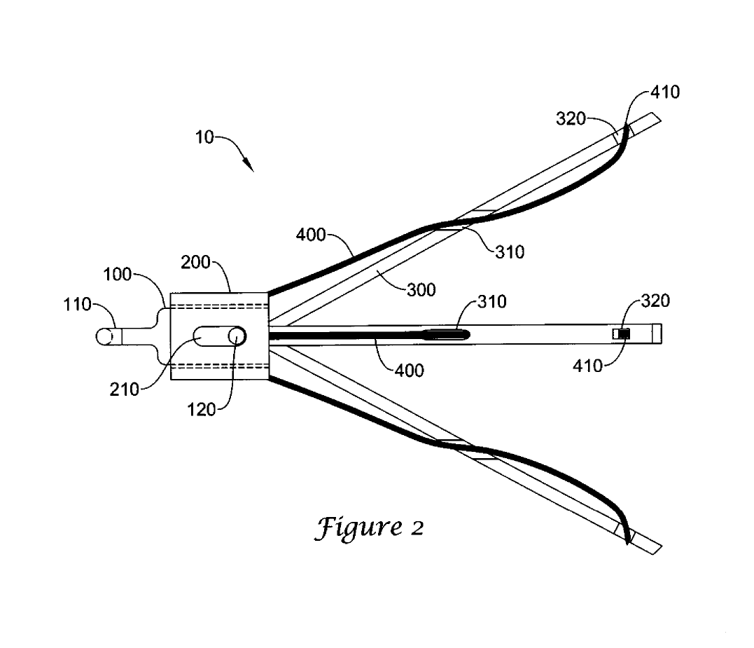

FIG. 1 illustrates an embodiment of an implantable filter 10 having a plurality of filter legs 300 attached to a first generally apical hub 100. As used herein, the terms “leg” and “legs” are used to indicate a predominantly solid shaft having a high length to width ratio. Although four filter legs 300 are illustrated, it will be appreciated that 3, 5, 6, 7, 8 or even more legs may be employed in each of the filters 10 described. The filter legs 300 have been illustrated as generally straight; however it will be appreciated that the filter legs 300 may be formed in any shape commonly employed for that purpose. For example, they may optionally be curved, include offset portions, and the like. Filter legs 300 may have any of a variety of cross-sectional shapes, such as circular, oval, rectangular, polygonal, and the like. The cross-sectional shape may vary along the filter leg 300 and shape transitions, if present, between cross-sectional shapes may be gradual or step-wise. Although the filter legs 300 have been illustrated as being of substantially equal length, it will be appreciated that filter legs 300 of somewhat different length may provide a benefit related to minimizing the diameter within which the implantable filter 10 may be compressed for delivery. This may be particularly the case if at least some of the filter legs 300 are provided with enlarged distal ends as discussed below. In other embodiments, filter legs 300 of somewhat different lengths may provide a benefit related to positioning the filter asymmetrically within the lumen.

The filter legs 300 may be fabricated separately and attached to a separate first generally apical hub 100 or the first generally apical hub 100 and filter legs 300 may be formed as an integral unit from a hypotube by laser cutting, etching, and the like. The filter legs 300 and the first apical hub 100 may be formed from materials commonly used for that purpose such as nickel titanium alloy, stainless steel, biocompatible polymers, and the like. The filter legs 300 may optionally be biased to self-expand upon deployment or may be actuated to expand by mechanical or thermal means.

In some embodiments, the first generally apical hub 100 includes a hook 110, illustrated in FIG. 1A, which facilitates deployment and/or retrieval of the implantable filter. In other embodiments, hook 110 is replaced by an alternate structure which may be grasped, pushed, pulled, twisted, or otherwise manipulated during deployment and/or retrieval of the implantable filter. In either event, the first generally apical hub may be moved axially relative to a second generally apical hub 200. The relative movement of the two generally apical hubs may be limited by suitable structures, as herein illustrated by the non-limiting example of aperture 210 and pin 120. The second generally apical hub 200 has attached thereto a plurality of wires 400 having distal ends 410 adapted to at least partially penetrate the walls of a vessel in which the implantable filter is deployed. Although the discussion to follow will focus on an implantable filter which may be deployed in a blood vessel, it will be appreciated that such filters may be implanted in lumens of other descriptions, such as catheters, ureters, and the like.

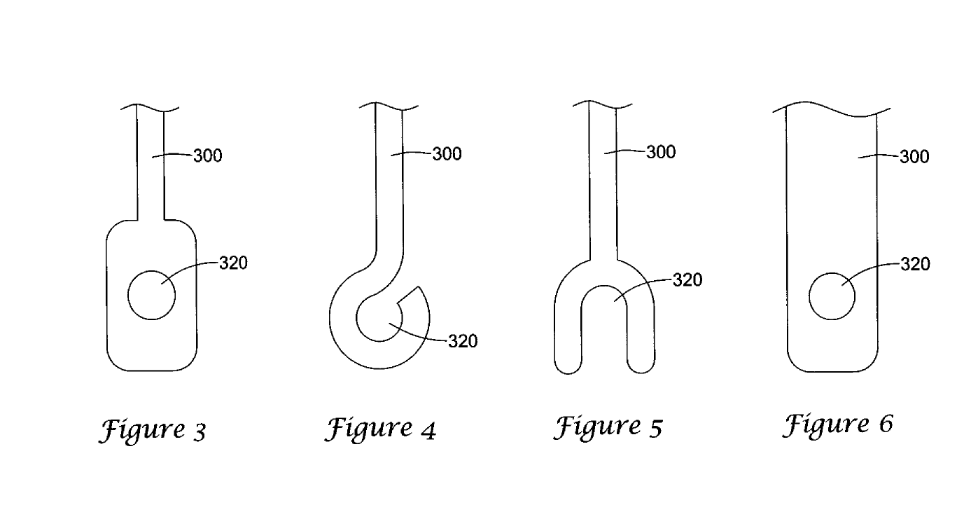

Each wire 400 is each disposed generally along and somewhat parallel to one of the plurality of filter legs 300 and passes from one side of the leg to the other before passing through a distal opening 320. In some embodiments, a wire 400 may pass transversely through or alongside its associated leg two or more times between the first generally apical hub and the distal end of the leg. For example, a wire 400 may pass from a position radially outside of the leg through an aperture 310 in an intermediate region of said leg 300 and thence along the leg 300 to and through a second aperture 320 to extend radially outward through the leg 300 forming an anchoring barb 410 as illustrated in FIG. 2. In other embodiments, the wire 400 may initially be located radially inward from the associated leg 300 and pass twice through or alongside the leg 300 before passing again radially outward through the leg to form anchoring barb 410. Alternate embodiments of the anchoring barb 410 will be discussed below. As used herein, the terms “wire” and “wires” are used to indicate a predominantly solid shaft having a high length to width ratio. Wires 400 may have any of a variety of cross-sectional shapes, such as circular, oval, rectangular, polygonal, and the like. The cross-sectional shape may vary along the wire 400 and transitions, if present, between cross-sectional shapes may be gradual or step-wise. In some embodiments, the cross-sectional shape of the wire 400 may be selected to interact with the shape of one or both of the distal opening 320 and aperture(s) 310 of the intermediate region to direct the longitudinal motion of the wire 400 relative to the associated leg 300 in a desired direction such as toward or away from a generally parallel orientation or to impart a torque to the wire 400.

The term “wire” is to be interpreted as including structures comprising two or more components which, when joined, provide the features of a wire 400 as described herein. Wires 400 and the second apical hub 200 may comprise materials commonly used for those purposes such as nickel titanium alloy, stainless steel, biocompatible polymers, and the like. In many embodiments the distal tip 410 may be formed from a material which minimizes the adhesion of the wire tip 410 to the lumen wall. The adhesion minimizing material may be either the material from which the wire tip 410 is formed or a coating (not shown) thereon.

Axial movement of the first generally apical hub 100 relative to the second generally apical hub 200 may cause the wire 400 and distal tip 410 to retract relative to the associated leg 300 thereby withdrawing the distal tip 410 from the wall of the lumen in which the filter is deployed. In some embodiments, the distal tip 410 will be withdrawn from the lumen wall along the axis of the wire tip 410 thereby minimizing the extraction force required. In other embodiments, the distal tip 410 may be adapted to pivot to withdraw from the lumen wall. In yet other embodiments, the distal tip 410 may both withdraw axially while pivoting to a degree. In some embodiments, the leg 300 may include an enlarged region which may serve as a landing pad to limit the penetration of the wire tip 410 and to reduce the likelihood of the distal end of the filter legs 300 becoming engulfed by tissue overgrowth or other deposits.

It will be appreciated that the filter 10 of these embodiments may be deployed with the wire tip(s) 410 in either the first extended position or the second retracted position. If the filter 10 is deployed with wire tips 410 in the first position, the outward expansion of the filter legs 300 may serve to anchor the wire tip 410 in the lumen wall. In the alternative, the filter 10 may be deployed in the second retracted position and the wire tip(s) 410 may subsequently be extended to engage the lumen wall by moving first generally apical hub 100 relative to second generally apical hub 200. If or when it becomes desirable to remove the filter 10 from engagement with the lumen wall, either permanently or for repositioning, the wire tip(s) 410 may subsequently be retracted to disengage from the lumen wall by moving first generally apical hub 100 relative to second generally apical hub 200 in the opposite sense.

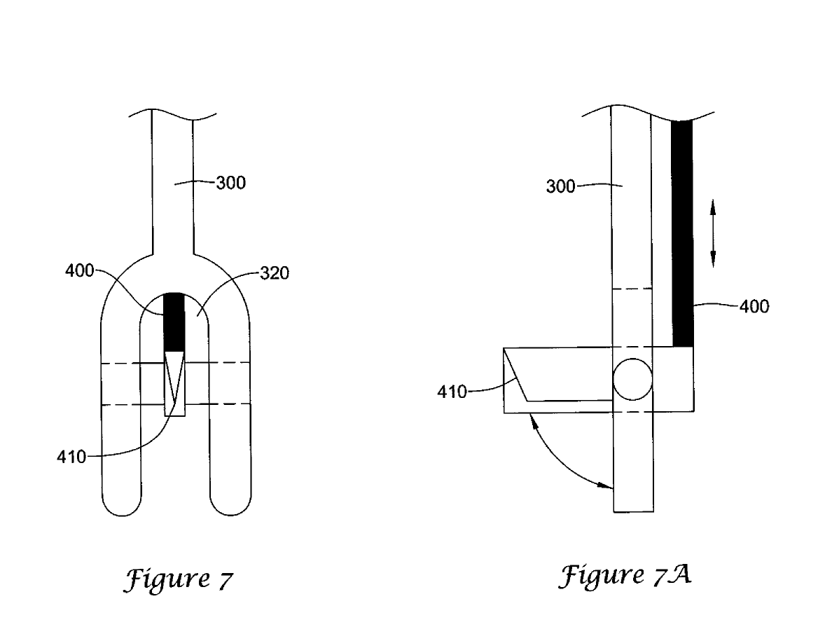

A number of non-limiting configurations of distal ends of filter legs 300 are illustrated in FIGS. 3-6, 7, 7A, 8 and 8A. In some embodiments, the leg ends depicted in FIGS. 3-6, 7, 7A, 8 and 8A may be formed as linear extensions of the filter legs 300. In other embodiments, the leg ends may be bent to lie approximately parallel to the vessel wall in a deployed configuration.

FIG. 3 illustrates a filter leg 300 including simple enlarged landing pad defining a through aperture 320. FIG. 4 illustrates an embodiment in which the filter legs 300 are formed from wires and the distal end of leg 300 has been formed into a loop which defines aperture 320. The loop may be open or closed. FIG. 5 illustrates a filter leg 300 in which the distal end of the leg 300 includes a slot-like aperture 320 which, depending on the motion imparted by the relative movement of the first and second hubs 100, 200 may be open or may be somewhat narrower at the distal end. If, for example, the distal tip 410 of wire 400 includes a curved portion which curves radially outward and somewhat proximally, the resulting forces may tend to hold the wire tip 410 within the slot near its proximal end. Partial closure of the distal end of slot-like aperture 320, may prevent the distal wire tip 410 from leaving the slot-like aperture 320, during manipulation. Upon withdrawal of the filter 10 from the lumen in which it is disposed, the wire tip 410 may move somewhat toward the distal end of the slot as it is withdrawn from the lumen wall. FIG. 6 illustrates a filter leg 300 in which the leg 300 maintains a generally uniform width in the distal region.

FIGS. 7 and 7A illustrate a multi-component wire 400 end in which the distal tip 410 assumes the form of a pivoting bar which optionally may include a sharpened edge to facilitate extraction of the distal tip 410 from the tissue in which it is embedded during deployment.

FIGS. 8 and 8A illustrate an alternate multi-component wire 400 end in which the distal tip 410 is a generally straight barb which includes one or more elements 420 which serve to prevent the distal wire tip 410 from extending excessively from the filter leg 300. In some embodiments, the element(s) 420 may resist an outward force provided by the configuration of wire 400 by bearing against the inner surface of the leg 300 while still allowing the distal tip 410 to pass through aperture 320 to engage the lumen wall.

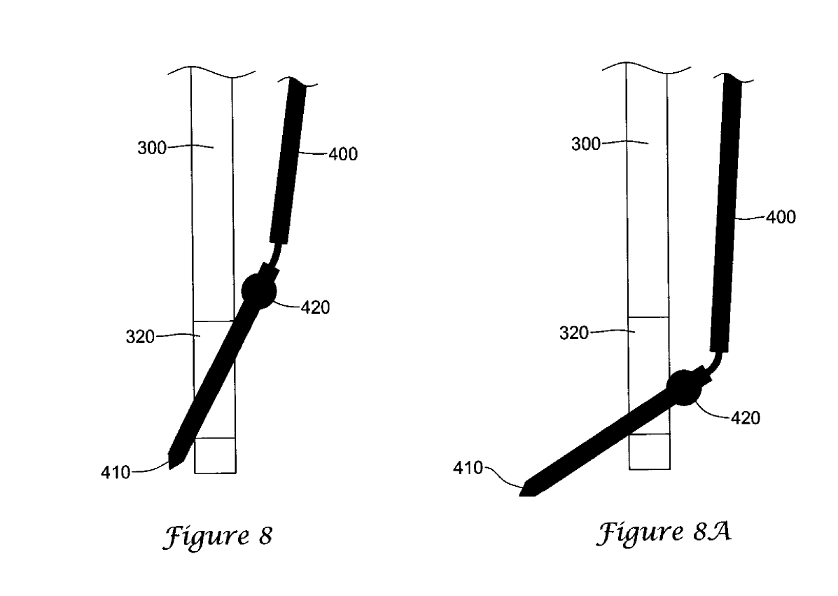

FIGS. 9-12 illustrate representative non-limiting configurations of the intermediate region of the legs of the implantable filter of FIGS. 1 and 2. FIG. 9 illustrates a simple aperture 310 through filter leg 300. The aperture 310, like those described below, may be repeated within the intermediate region of the filter leg 300. Although the aperture 310 is depicted as round, it may assume any convenient shape. As mentioned above, the shape may be selected to interact with the shape of the wire 400 which passes therethrough. FIG. 10 represents a portion of a leg 300 which has been twisted into a helical arrangement which allows the wire 400 to pass through the center of the helix. In such embodiments, the wire may be viewed as passing from one side of the leg 300 to the second side of the leg 300 and back again depending on the number of turns of the helix. It will be appreciated that a similar guiding effect may be attained by arranging for the wire 400 to twine around the leg 300 in the intermediate region of the leg 300. Similarly, when the leg 300 is formed of wire, the wire leg 300 may twine around the wire 400 in the intermediate region. FIG. 11 illustrates an aperture 310 which penetrates the leg 300 at an acute angle to direct the wire 400 along a path which is more nearly parallel to the surface of the leg 300 in the intermediate region. FIG. 12 illustrates an alternate arrangement in which the wire 400 passes the filter leg 300 through a short tubular element 312 which is fixedly attached to the side of the leg 300. Tubular element 312 provides the aperture 310 which controls the passage of wire 400 through the intermediate region of the leg. In some embodiments, the tubular element 312 may lie generally parallel to filter leg 300 and, for the purposes of this description, this arrangement will be considered to provide a transverse passage relative to the filter leg 300. Such a passage may be viewed as extending from the inside of the filter leg 300 to the outside or from the outside of filter leg 300 to the inside.

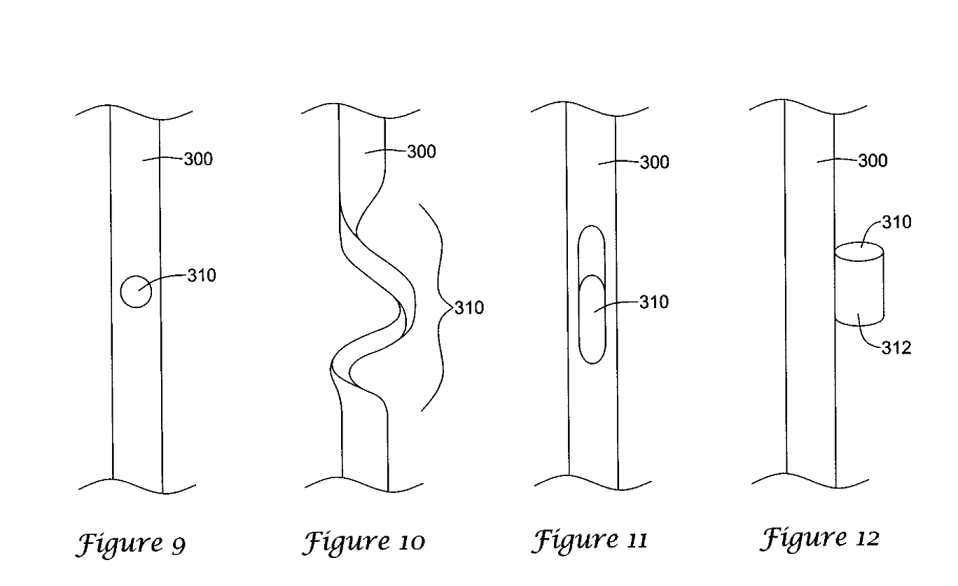

FIG. 13 illustrates a filter leg 300 having a zigzag configuration which is generally believed to have superior filtering capability. In this embodiment, multiple tubular elements 312 have been disposed along the side of the filter leg 300 to provide multiple apertures 310 disposed therealong. In this embodiment, as discussed above, the tubular elements 312 are to be viewed as providing a transverse passage of wire 400 relative to filter leg 300 and a distal portion 460 of leg 400 may be directed inward prior to passing outward through aperture 320 to engage the lumen wall.

In other embodiments, the alternating diagonal segments of filter leg 300 may direct the intervening longitudinal segments toward and away from the lumen wall and the tubular elements 312 may be disposed along the diagonal segments to allow the generally straight wire 400 to weave alternately between the inner and outer surface of the leg 300 before passing outward through aperture 320 to engage the lumen wall. In yet other embodiments, one or more of the illustrated tubular elements 312 may be omitted and the leg 300 may be viewed as twining about wire 400 to create an extended intermediate region.

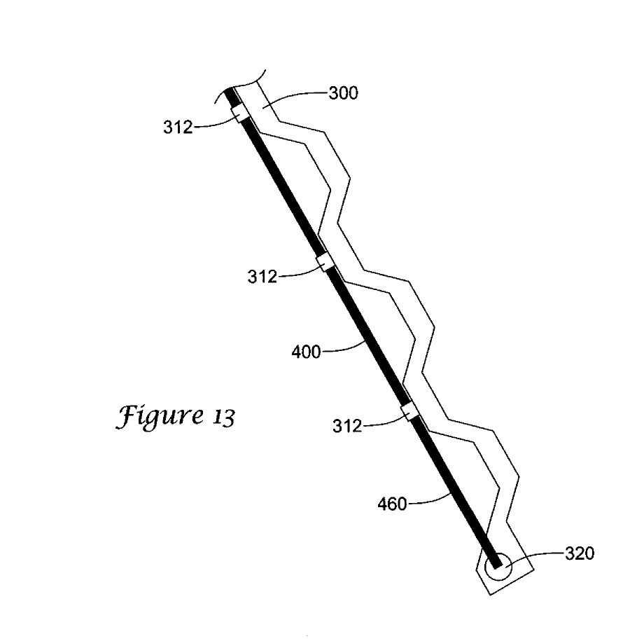

FIG. 14 illustrates a filter of FIGS. 1 and 2 to which additional centering struts 340 have been added. In some embodiments, centering struts 340 may be provided with barbs or with a set of wires (not shown) which function in the manner of the wires 400 associated with filter legs 300.

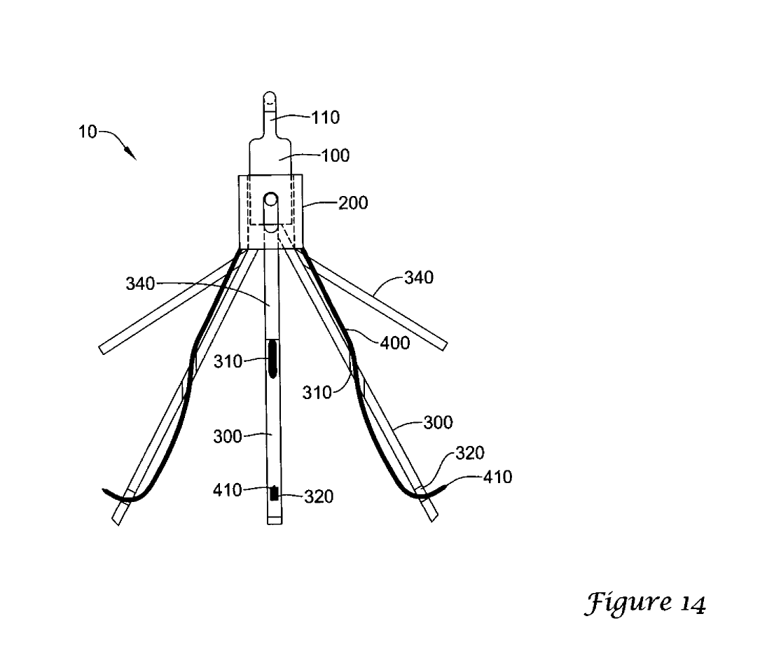

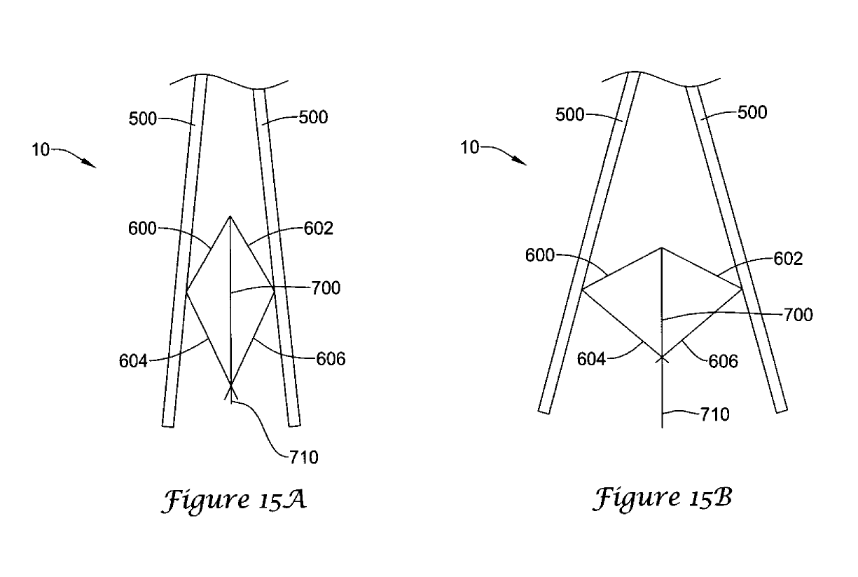

FIGS. 15A and 15B illustrate a portion of an embodiment in which the struts 600, 602, 604, and 606 provide the guiding function of the apertures 310 described above and the join of struts 604 and 606 optionally are somewhat extended at their join to provide a landing pad. Wire 700 and its distal end 710 is slidingly received at the join between the ends of legs 604 and 606. In these embodiments, each pair of legs 500 may include therebetween struts 600, 602, 604, and 606 or the struts 600, 602, 604, and 606 may be disposed between only some pairs of adjacent legs 500. Only a portion of one pair of legs 500 is shown in the figures to reduce clutter. In these embodiments, wires 700 are attached to the joins between struts 600 and 602 which serve collectively to provide the second apical head of the earlier embodiments. The remaining ends of struts 600 and 604, as well as struts 602 and 606, are attached to filter legs 500 in an intermediate region thereof. The paired struts 600 and 604, as well as struts 602 and 606, may be joined to legs 500 at a single point within the intermediate region or they may be spaced somewhat apart. The joins between the struts and between the struts and the legs are configured to provide a degree of flexure within the plane of a group of struts 600, 602, 604, and 606. This arrangement causes the join between struts 600, 602 and wire 700 to be spaced farther from the join between struts 602 and 606 when legs 500 are in a first configuration in which they are substantially parallel and to be spaced closer together in a second configuration in which at least some of the plurality of legs 500 are radially expanded relative to a central axis passing through the first generally apical hub (not shown). This in turn causes the distal end 710 of wire 700 to extend from the join of struts 604, 606 to create an anchoring distal tip 710 which may engage the lumen wall within which the filter is disposed. In some embodiments, the distal tip 710 may curve outward from the plane defined by the adjacent filter legs 500 and their associated struts 600, 602, 604, and 606 when the filter legs 500 are radially expanded relative to a central axis passing through the first generally apical hub (not shown).

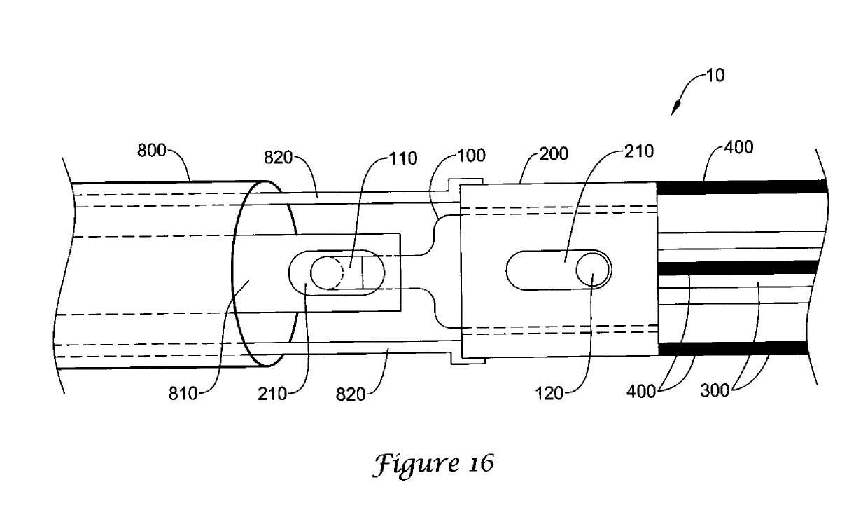

In another aspect, the disclosure relates to an implantable filter system, as partially depicted in FIG. 16, comprising for example a filter 10 as discussed above, a catheter 800 sized and adapted at its distal end to contain at least a portion of a collapsed configuration of filter 10, a releasable actuator 810 adapted to engage a portion of filter 10, such as hook 110 of the embodiment of FIGS. 1 and 2, and a manipulation element 820 adapted to eject the implantable filter 10 from the catheter or to withdraw at least a portion of the implantable filter 10 within the distal region of the catheter 800. For example, releasable actuator 810 may be adapted to engage hook 110 of first generally apical hub 100 while a manipulation element 820, tubular element 820 or separate elements 820, engage the second generally apical hub 200. Following ejection of filter 10 from catheter 800 by distal motion of tubular element 820, or separate elements 820, thereby deploying filter 10 with legs 300 and wires 400 in the second position, as illustrated in FIG. 16, the legs 300 may be expanded to contact the wall of the lumen in which the implantable filter 10 is to be deployed. Retraction of releasable actuator 810 relative to manipulation element 820 will cause distal tips 410 of wires 400 to extend from distal openings 320 to engage the lumen wall. In the alternative, the filter 10 may be advanced from catheter 800 with actuator 810 retracted relative to manipulation element 820 thereby allowing the extended distal tips 410 of wires 400 to be extended relative to distal openings 320 in filter legs 300 as the filter 10 deploys to allow the extended distal tips 410 to strike the lumen wall as the legs 300 expand. In either event, releasable actuator 810 may be advanced relative to manipulation element 820, and optionally twisted, to release hook 110 from releasable actuator 810, whereupon releasable actuator 810, manipulation element 820, and catheter 800 may be removed.

When it is desirable to remove or reposition implantable filter 10, catheter 800 may be advanced to a position just proximal of the proximal end of implanted filter 10. In some embodiments, catheter 800 may include alignment elements (not shown) to ensure alignment of catheter 800 with implanted filter 10. For example, the distal end of catheter 800 may include radially expansible guides which locate the first apical head. In some embodiments, separate elements of manipulation element 820 may provide that function when extended sufficiently from the distal end of catheter 800. Releasable actuator 810 may then be advanced to engage hook 110 of the embodiment of FIGS. 1 and 2 or equivalent engagable structure or structures of other embodiments of the invention. The manipulation element 820 may then be retracted until it engages the second generally apical hub 200 of the embodiment of FIGS. 1 and 2, if desired, thereby allowing the second generally apical hub 200 of the embodiment of FIGS. 1 and 2 to be with drawn relative to releasable actuator 810 to retract wires 400, and distal tips 410 relative to apertures 320 prior to withdrawing implantable filter 10 at least partially within catheter 800 thereby collapsing the filter legs 300 to a first generally parallel configuration for removal or repositioning. In some embodiments, the movement of first generally apical hub 100 relative to second generally apical hub 200 may partially or completely collapse legs 300 prior to withdrawal of filter 10 to a position within the distal end of catheter 800. If desired, the deployment steps may be repeated in a new location within the lumen containing the liquid to be filtered.

Although the illustrative examples described above relate to a filter implantable in a body lumen, additional applications are also contemplated. In such an embodiment, the size of the filter may be adjusted to correspond to the size of the lumen of, for example, a piece of tubing or a pipe. Further, although the exemplary filters discussed above have been illustrated as having configurations in which the legs expand symmetrically to position the filter such that its central axis generally coincides with the central axis of the lumen in which it is deployed, it is to be understood that embodiments in which one or more legs of the filter are disposable generally parallel to and potentially in contact with the lumen wall such that the central axis of the filter lies at an angle to the central axis of the lumen are also contemplated.

In addition to the elements discussed above, the implantable filters may include other elements commonly employed in such filters. For example, the filter and/or the catheter portion of the system may include one or more radiopaque markers. Similarly, in some embodiments, the filter may include a filter membrane, functional coatings, and the like.

Various modifications and alterations of this invention will become apparent to those skilled in the art without departing from the scope and principles of this invention, and it should be understood that this invention is not to be unduly limited to the illustrative embodiments set forth hereinabove. All publications and patents are herein incorporated by reference to the same extent as if each individual publication or patent was specifically and individually indicated to be incorporated by reference.

What is claimed is:

1. An implantable filter comprising:
a first generally apical hub;
a first plurality of solid legs each having a proximal end, a distal end region, and an intermediate region therebetween, said first plurality of solid legs being fixedly attached to the first generally apical hub and having a first configuration in which the first plurality of solid legs are substantially parallel and a second configuration in which the distal ends of at least some the first plurality of solid legs are radially expanded relative to a central axis passing through the first generally apical hub; and
a second plurality of wires each having distal ends and proximal ends, said wires of the second plurality of wires being fixedly attached at their proximal ends to a second generally apical hub which is slidably moveable relative to the first generally apical hub,
wherein each of the second plurality of wires is individually associated with one of the solid legs of the first plurality of solid legs at the intermediate region thereof and distal end region thereof,
further wherein the association of each wire with the solid leg at the distal end region thereof comprises the wire passing transversely through the distal end region of the solid leg such that each wire has a first position in which the distal end of the wire protrudes radially outward from the distal end region of the solid leg and a second position in which the wire does not protrude significantly radially outward from the distal end region of the solid leg.

2. The implantable filter of claim 1, wherein the association of each of the second plurality of wires with a solid leg at the intermediate region thereof comprises the wire passing at least once transversely therethrough.

3. The implantable filter of claim 1, wherein the individual association of each of the second plurality of wires with the solid leg at the intermediate region thereof comprises one of the wire and the solid leg being twined about the other.

4. The implantable filter of claim 1, wherein the distal end of each of the second plurality of wires comprises a sharpened tip.

5. The implantable filter of claim 1, wherein the distal region of the solid leg includes a landing pad.

6. The implantable filter of claim 1, wherein the first generally apical hub and the solid legs are formed from a single tubular element by the removal of portions thereof.

7. The implantable filter of claim 1, wherein the first generally apical hub and the plurality of solid legs comprise one of a nickel titanium alloy, stainless steel, and a biocompatible polymer.

8. The implantable filter of claim 1, wherein the second plurality of wires comprise one of a nickel titanium alloy, stainless steel, and a biocompatible polymer.

9. The implantable filter of claim 1, further comprising a radiopaque marker.

10. The implantable filter of claim 1, further comprising an engagable element adapted to effect relative axial movement of the first generally apical hub relative to the second generally apical hub.

11. The implantable filter of claim 1, further comprising an additional plurality of solid legs and an additional plurality of wires associated and configured relative to each other in the manner of claim 1.

12. The implantable filter of claim 11, wherein the additional plurality of solid legs and the additional plurality of wires are fixedly attached to the first generally apical hub and the second generally apical hub respectively.

13. The implantable filter of claim 11, wherein at least one of the additional plurality of solid legs and the additional plurality of wires is fixedly attached to a generally apical hub other than the first generally apical hub and the second generally apical hub.

14. An implantable filter system comprising:
a filter of claim 1;
a catheter sized and adapted to contain at least a portion of the filter of claim 1 within the distal region thereof;
a releasable actuator adapted to engage a portion of the filter of claim 1 and to effect relative axial movement of the first generally apical hub relative to the second generally apical hub; and
a manipulation element adapted to eject the implantable filter from the catheter or to withdraw at least a portion of the implantable filter within the distal region of the catheter.

15. An implantable filter comprising:
a generally apical hub;
a plurality of legs each having a proximal end, a distal end region, and an intermediate region therebetween, said legs being fixedly attached to the generally apical hub and having a first configuration in which the legs are substantially parallel and a second configuration in which the distal ends of at least some the plurality of legs are radially expanded relative to a central axis passing through the apical hub;
a pair of first struts attached to a first leg and a second leg of the plurality of legs within the respective intermediate regions of the first and second legs and extending to a first join proximal to their distal ends;
a pair of second struts attached to a first leg and a second leg within the respective intermediate regions of the first and second legs and extending to a second join distal to their proximal ends; and
a wire extending from the first join and slidingly received through an aperture associated with the second join,
wherein upon radial expansion of the first and second leg to the second configuration the first join and the second join are configured to move generally toward one another whereby the wire protrudes from the second join in a direction radially outward from the central axis passing through the apical hub.

16. The implantable filter of claim 15, further comprising an additional pair of first struts, an additional pair of second struts, and an additional wire disposed between at least one additional pair of legs.

17. The implantable filter of claim 16, wherein each pair of legs comprises an additional pair of first struts, an additional pair of second struts, and an additional wire disposed therebetween.

18. A method of deploying a filter of claim 1 comprising:
positioning the filter of claim 1 within a distal region of a catheter;
positioning the distal region of the catheter within a lumen containing a fluid to be filtered;
ejecting the filter of claim 1 from the catheter thereby allowing at least some the distal ends of the plurality of solid legs to become radially expanded relative to a central axis passing through the apical hub; and
implanting the distal ends of the plurality of wires in a wall of the lumen containing a fluid to be filtered.

19. The method of claim 18, wherein the implanting step comprises the moving the first generally apical hub of the filter of claim 1 axially relative to the second generally apical hub, thereby advancing the plurality of wires relative to the plurality of solid legs while moving the plurality of wires from second position in which the wires do not protrude significantly radially outward from the distal end region of the solid leg to a first position in which the distal ends of the wires protrude radially outward from the distal end regions of the solid legs thereby at least partially penetrating the wall of the lumen.

20. A method of retrieving a filter of claim 1 comprising:
positioning a distal region of a catheter proximally adjacent to a first generally apical hub of the filter of claim 1;
moving the first generally apical hub of the filter of claim 1 axially relative to the second generally apical hub, thereby withdrawing the plurality of wires relative to the plurality of solid legs thereby moving the plurality of wires from first position in which the wires protrude significantly radially outward from the distal end region of the solid legs to a second position in which the distal ends of the wires do not significantly protrude radially outward from the distal end region of the solid legs thereby at least partially withdrawing the wire from the wall of the lumen;
withdrawing the filter of claim 1 at least partially within a distal region of the catheter; and
withdrawing the catheter and the filter of claim 1 from the lumen.
